(19) United States
(12) Patent Application Publication
(10) Pub. No.: US 2008/0069905 A1
(43) Pub. Date: Mar. 20, 2008

(54) Device for application of medicaments, manufacturing method therefor, and method of treatment

(76) Inventor: Tor Peters, Helsingborg (SE)

Correspondence Address: KNOBBE MARTENS OLSON & BEAR LLP, 2040 MAIN STREET, FOURTEENTH FLOOR, IRVINE, CA, 92614, US

(21) Appl. No.: 11/891,671

(22) Filed: Aug. 10, 2007

Related U.S. Application Data
(63) Continuation of application No. PCT/EP06/50890, filed on Feb. 13, 2006.
(60) Provisional application No. 60/652,759, filed on Feb. 14, 2005.
(60) Provisional application No. 60/666,501, filed on Mar. 30, 2005.
(60) Provisional application No. 60/711,006, filed on Aug. 24, 2005.

(30) Foreign Application Priority Data
Feb. 11, 2005 (EP) 05002936.2
Feb. 11, 2005 (EP) 05006463.3
Aug. 23, 2005 (EP) 05018269.0

(51) Int. Cl. A61K 33/00; A61M 35/00; A61P 17/02

(52) U.S. Cl. 424/718; 604/293; 604/304

(57) ABSTRACT

A therapeutic treatment device is provided, which comprises a compound comprising a drug and a nitric oxide (NO) eluting polymer arranged to contact a treatment site in or on a body. The device is acting as a booster for drug eluting patches, e.g. pharmaceuticals, vitamins, nicotin, nitroglycerin, whereby with advantage two therapeutic treatments, of significant value, are combined in one treatment. A synergetic effect is achieved by such devices because NO that is eluted from the device boosts the effect of the drug, as the treatment site is more susceptible to said drug by the effect of the eluted NO.

### RELATED APPLICATIONS

This application is a continuation of PCT/EP2006/050890, filed Feb. 13, 2006, which claims priority to European Patent Application No. 05002936.2, filed Feb. 11, 2005; European Patent Application No. 05006463.3, filed Feb. 11, 2005; U.S. Provisional Application No. 60/652,759, filed Feb. 14, 2005; U.S. Provisional Application No. 60/666,501, filed Mar. 30, 2005; European Patent Application No. 05018269.0, filed Aug. 23, 2005; and U.S. Provisional Application No. 60/711,006, filed Aug. 24, 2005. The entire content of these applications is incorporated herein by reference.

### BACKGROUND OF THE INVENTION

1. Field of the Invention

This invention pertains in general to an improved device for application of medicaments, and in particular a device for the application of medicaments with a vasoconstrictive effect, such as a side effect. More particularly the invention relates to a process of manufacturing of such an improved device, wherein said device and process of manufacturing involve the use of nitric oxide (NO), as well as a corresponding method of treatment.

2. Description of the Related Art

Medicaments, drugs or pharmaceuticals of today may differ widely in respect of intended effect, therapeutic field or use, or target area. Some of these medicaments, drugs, or pharmaceuticals are accompanied with more or less adverse side effects. Some medicaments, drugs, or pharmaceuticals are accompanied with the side effect of being vasoconstrictive, some are intended to be vasoconstrictive for a period of time, and some would be advantageously effected by a vasodilating effect.

One example of a medicament, drug, or pharmaceutical, with a side effect of being vasoconstrictive is nicotine. Nicotine is used in patches of different kinds, and chewing gums, to provide smokers with small amounts of the addictive substance in treatment to quit smoking. Since nicotine is vasoconstrictive, this effect prevents the active substance (nicotine itself) from reaching and stimulating the target areas in curing of smoking. Other examples of active substances with the side effect of being vasoconstrictive, and that would be positively affected by a vasodilating effect is diclofenac (Cataflam® and Voltaren®), and cortisone.

Other medicaments, drugs, or pharmaceuticals have the ability to be absorbed through the skin. These medicaments, drugs, or pharmaceuticals may be more or less prone to absorption through skin, and would in all cases be positively affected by a device that would present an increased absorption.

It is known that nitric oxide (NO) provides an alternative to conventional therapies, such as antibiotics. Nitric oxide is a highly reactive molecule that is involved in many cell functions. In fact, nitric oxide plays a crucial role in the immune system and is utilized as an effector molecule by macrophages to protect itself against a number of pathogens, such as fungi, viruses, bacteria etc., and general microbial invasion.

NO is also known to have an anti-pathogenic, especially an anti-viral, effect, and furthermore NO has an anti-cancerous effect, as it is cytotoxic and cytostatic in therapeutic concentrations, i.e. it has among other effects tumoricidal and bacteriocidal effects. NO has for instance cytotoxic effects on human hematological malignant cells from patients with leukemia or lymphoma, whereby NO may be used as a chemotherapeutic agent for treating such hematological disorders, even when the cells have become resistant to conventional anti-cancer drugs.

However, due to the short half-life of NO, it has hitherto been very hard to treat viral, bacteria, virus, fungi or yeast infections with NO. This is because NO is actually toxic in high concentrations and has negative effects when applied in too large amounts to the body. NO is actually also a vasodilator, and too large amounts of NO introduced into the body will cause a complete collapse of the circulatory system. On the other hand, NO has a very short half-life of fractions of a second up to a few seconds, once it is released. Hence, administration limitations due to short half-life and toxicity of NO have been limiting factors in the use of NO in the field of anti-pathogenic and anti-cancerous treatment so far.

In recent years research has been directed to polymers with the capability of releasing nitrogen oxide when getting in contact with water. Such polymers are for example polyalkyleneimines, such as L-PEI (Linear PolyEthyleneImine) and B-PEI (Branched PolyEthyleneImine), which polymers have the advantage of being biocompatible.

Other examples for NO eluting polymers are given in U.S. Pat. No. 5,770,645, wherein polymers derivatized with at least one —NO_{X} group per 1200 atomic mass unit of the polymer are disclosed, X being one or two. One example is an S-nitrosylated polymer and is prepared by reacting a polythiolated polymer with a nitrosylating agent under conditions suitable for nitrosylating free thiol groups.

Akron University has developed a NO-eluting L-PEI molecule that can be nano-spun onto the surface of medical devices to be permanently implanted in the body, such as implanted grafts, showing significant improvement of the healing process and reduced inflammation when implanting such devices. According to U.S. Pat. No. 6,737,447, a coating for medical devices provides nitric oxide delivery using nanofibers of linear poly(ethylenimine)-diazeniumdiolate. Linear poly(ethylenimine)diazeniumdiolate releases nitric oxide (NO) in a controlled manner to tissues and organs to aid the healing process and to prevent injury to tissues at risk of injury. Electrospun nanofibers of linear poly(ethylenimine)diazeniumdiolate deliver therapeutic levels of NO to the tissues surrounding a medical device while minimizing the alteration of the properties of the device. A nanofiber coating, because of the small size and large surface area per unit mass of the nanofibers, provides a much larger surface area per unit mass while minimizing changes in other properties of the device.

US 2002/0012816 discloses hydrogels, comprising macromers, with the ability to release nitric oxide. Examples of these macromers are PVA and PEG. The polymers may serve as carriers for biologically active materials, such as therapeutic, prophylactic, or diagnostic agents. Nothing is mentioned in US 2002/0012816 about boosting an effect of a pharmaceutical with nitric oxide, only that nitric oxide is used together with therapeutic agents.

WO 2004/012659 discloses proton pump inhibitors, such as omeprazole, pantoprazole and paniprazole, optionally in combination with a compound with the possibility to donate, transfer, or release nitric oxide. WO 2004/012659 does not describe nitric oxide eluting polymers, but nitric oxide eluting compounds, such as S-nitroso-polypeptides.

WO 01/85013 discloses vasoactive agents, such as potassium channel activators, dopamine agonists, and thromboxane inhibitors, optionally in combination with a compound with the possibility to donates, transfers, or releases nitric oxide. Such a nitrogen oxide releasing compound can for example be S-nitrosothiol. WO 01/85013 does not describe nitric oxide eluting polymers, but nitric oxide eluting compounds, such as S-nitroso-polypeptides, to improve the action of the vasoactive agents.

US 2002/0082221 discloses a nitric oxide releasing S-nitrosylated, N-nitrosylated, and/or O-nitrosylated lipid and administration methods thereof. The lipid of US 2002/0082221 is not a nitric oxide eluting polymer.

WO 2004/012874 discloses a nitric oxide releasing medical device. The device comprises a substrate with a metallic surface, to which an amine-functionalized silane residue can be bound. Nitric oxide may be bonded to said amine-functionalized silane residue.

WO 03/092763 discloses nanotubules with the ability to bind nitric oxide or gas with nitric oxide like properties. WO 03/092763 describes that pharmaceuticals may be used in combination with said nanotubules. WO 03/092763 does not describe a nitric oxide eluting polymer, but nanotubules containing nitric oxide.

However, the disclosure is both silent concerning an improvement of present technology in respect of improving absorption, counteracting vasoconstriction, and providing vasodilation, of topically active medicaments, drugs, and/or pharmaceuticals.

Hence, an improved, or more advantageous, device for the cooperation between drugs and NO is needed in the art. It is desired that said device provides counteraction of side effects, in the form of vasoconstrictive effect, of said drugs, provides an increased absorption of said drugs, and boosts the effect of said drugs.

### SUMMARY OF THE INVENTION

Accordingly, the present invention preferably seeks to mitigate, alleviate or eliminate one or more of the above-identified deficiencies in the art and disadvantages singly or in any combination and solves, among others, at least some of the problems mentioned above, by providing a device, a manufacturing method for the latter and a use of nitric oxide according to the appended patent claims.

According to one aspect of the invention, a device is provided that allows for cooperation between drugs, with a vasoconstrictive effect, or that would be positively affected by a vasodilating effect, and an nitric oxide (NO) eluting polymer, arranged to contact the area to be treated, such that a vasodilating effect and a boosting effect is accomplished by a therapeutic dose of nitric oxide when NO is eluted from said nitric oxide eluting polymer to said area.

According to another aspect of the invention, a manufacturing process for such a device is provided, wherein the process is a process for forming a device that allows for cooperation between drugs, with a vasoconstrictive effect, or that would be positively affected by a vasodilating effect, and a boosting effect, and an nitric oxide (NO) eluting polymer. The process comprises selecting a plurality of nitric oxide eluting polymeric particles, such as nanofibers, fibers, nanoparticles, or microspheres, and deploying said nitric oxide eluting particles in combination with drugs, with a vasoconstrictive effect, or that would be positively affected by a vasodilating effect, and a boosting effect, in form of a patch/pad or tape/coating to be comprised in said device. Alternatively the NO eluting particles, and the drugs, are admixed to an ointment, cream, foam, or gel.

The present invention has at least the advantage over the prior art that it provides target exposure of a vasoconstricted area, or an area that would be positively affected by a vasodilating action, to NO, whereby a very effective vasodilating therapy is achievable.

### BRIEF DESCRIPTION OF THE DRAWINGS

These and other aspects, features and advantages of which the invention is capable of will be apparent and elucidated from the following description of embodiments of the present invention, reference being made to the accompanying drawings, in which

FIG. 1 is a schematic illustration of a patch/pad 10 according to an embodiment of the invention, and

FIG. 2 is a schematic illustration of a tape/coating 20 according to an embodiment of the invention, and

FIG. 3 is a graph illustrating different elutions of nitric oxide from two polymer mixtures.

### DETAILED DESCRIPTION OF THE EMBODIMENTS

The following description focuses on embodiments of the present invention applicable to a device, in form of a patch/pad, which allows for cooperation between drugs, with a vasoconstrictive effect, or that would be positively affected by a vasodilating effect, and a boosting effect of said drug, and an nitric oxide (NO) eluting polymer, arranged to contact the area to be treated, such that a vasodilating effect is accomplished by a therapeutic dose of nitric oxide when NO is eluted from said nitric oxide eluting polymer to said area.

In the context of the present invention the term “drug” is to be interpreted as including all pharmaceuticals, active components, vitamins, nutrition agents, which may be used on a mammal body, such as a human body, to achieve a therapeutic, treating, healing, and/or curative effect.

With regard to nitric oxide (nitrogen monoxide, NO), its physiological and pharmacological roles have attracted much attention and thus have been studied. NO is synthesized from arginine as the substrate by nitric oxide synthase (NOS). NOS is classified into a constitutive enzyme, cNOS, which is present even in the normal state of a living body and an inducible enzyme, iNOS, which is produced in a large amount in response to a certain stimulus. It is known that, as compared with the concentration of NO produced by cNOS, the concentration of NO produced by iNOS is 2 to 3 orders higher, and that iNOS produces an extremely large amount of NO.

In the case of the generation of a large amount of NO as in the case of the production by iNOS, it is known that NO reacts with active oxygen to attack exogenous microorganisms and cancer cells, but also to cause inflammation and tissue injury. On the other hand, in the case of the generation of a small amount of NO as in the case of the production by cNOS, it is considered that NO takes charge of various protective actions for a living body through cyclic GMP (cGMP), such as vasodilator action, improvement of the blood circulation, antiplatelet-aggregating action, antibacterial action, anticancer action, acceleration of the absorption at the digestive tract, renal function regulation, neurotransmitting action, erection (reproduction), learning, appetite, and the like. Heretofore, inhibitors of the enzymatic activity of NOS have been examined for the purpose of preventing inflammation and tissue injury, which are considered to be attributable to NO generated in a large amount in a living body. However, the promotion of the enzymatic activity (or expressed amount) of NOS (in particular, cNOS) has not been examined for the purpose of exhibiting various protective actions for a living body by promoting the enzymatic activity of NOS and producing NO appropriately.

In recent years research has been directed to polymers with the capability of releasing nitrogen oxide when getting in contact with water. Such polymers are for example polyalkyleneimines, such as L-PEI (Linear PolyEthyleneImine) and B-PEI (Branched PolyEthyleneImine), which polymers have the advantage of being biocompatible. Another advantage is that NO is released without any secondary products that could lead to undesired side effects.

The polymer fibers may be manufactured by electro spinning, gas spinning, air spinning, wet spinning, dry spinning, melt spinning, and gel spinning. Electro spinning is a process by which a suspended polymer is charged. At a characteristic voltage a fine jet of polymer releases from the surface in response to the tensile forces generated by interaction by an applied electric field with the electrical charge carried by the jet. This process produces a bundle of polymer fibers, such as nanofibers. This jet of polymer fibers may be directed to a surface to be treated.

Furthermore, U.S. Pat. No. 6,382,526, U.S. Pat. No. 6,520,425, and U.S. Pat. No. 6,695,992 disclose processes and apparatuses for the production of such polymeric fibers. These techniques are generally based on gas stream spinning, also known within the fiber forming industry as air spinning, of liquids and/or solutions capable of forming fibers.

Other example for NO eluting polymers are given in U.S. Pat. No. 5,770,645, wherein polymers derivatized with at least one —NOX group per 1200 atomic mass unit of the polymer are disclosed, X being one or two. One example is an S-nitrosylated polymer and is prepared by reacting a polythiolated polymer with a nitrosylating agent under conditions suitable for nitrosylating free thiol groups.

Akron University has developed NO-eluting L-PEI molecule that can be nano-spun onto the surface of permanently implanted medical devices, such as implanted grafts, showing significant improvement of the healing process and reduced inflammation when implanting such devices. According to U.S. Pat. No. 6,737,447, a coating for medical devices provides nitric oxide delivery using nanofibers of linear poly(ethylenimine)-diazeniumdiolate. Linear poly(ethylenimine)diazeniumdiolate releases nitric oxide (NO) in a controlled manner.

However, the meaning of “controlled” in the context of U.S. Pat. No. 6,737,447 is only directed to the fact that nitric oxide is eluted from the coating during a period of time, i.e. that the nitric oxide not is not eluted all at once. Therefore, the interpretation of “controlled” in respect of U.S. Pat. No. 6,737,447 is different from the meaning of “regulating” in the present invention. “Regulate or control”, according to the present invention is intended to be interpreted as the possibility to vary the elution of nitric oxide to thereby achieve different elution profiles.

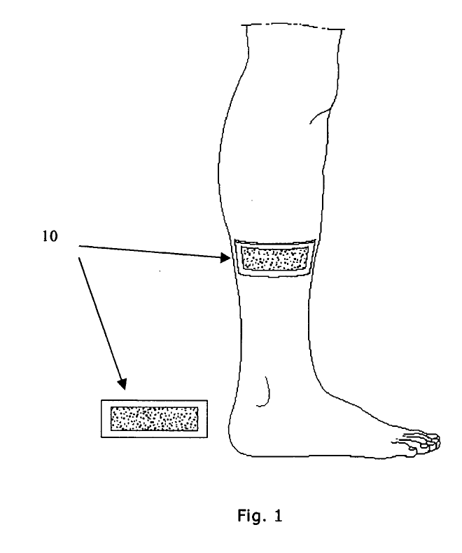

In an embodiment of the invention, according to FIG. 1, the device is in the form of a patch/pad, said patch/pad being covered on the inside with nanofilament of any of the NO-eluting polymers according to above, such as polyalkyleneimines, such as L-PEI (Linear PolyEthyleneImine) and B-PEI (Branched PolyEthyleneImine), which polymers have the advantage of being biocompatible. This patch/pad does also comprise nicotine in the therapeutic amount.

A polymer comprising an O-nitrosylated group is also a possible nitric oxide eluting polymer. Thus, in one embodiment, the nitric oxide eluting polymer comprises diazeniumdiolate groups, S-nitrosylated and O-nitrosylated groups, or any combinations thereof.

In still another embodiment said nitric oxide eluting polymer is a poly(alkyleneimine)diazeniumdiolate, such as L-PEI-NO (linear poly(ethyleneimine)diazeniumdiolate), where said nitric oxide eluting polymer is loaded with nitric oxide through the diazeniumdiolate groups and arranged to release nitric oxide at a treatment site.

Some other examples of a suitable nitric oxide eluting polymer are selected from the group comprising amino cellulose, amino dextrans, chitosan, aminated chitosan, polyethyleneimine, PEI-cellulose, polypropyleneimine, polybutyleneimine, polyurethane, poly(buthanediol spermate), poly(iminocarbonate), polypeptide, Carboxy Methyl Cellulose (CMC), polystyrene, poly(vinyl chloride), and polydimethylsiloxane, or any combinations of these, and these mentioned polymers grafted to an inert backbone, such as a polysaccharide backbone or cellulosic backbone.

In still another embodiment the nitric oxide eluting polymer may be a O-derivatized NONOate. This kind of polymer often needs an enzymatic reaction to release nitric oxide.

Other ways of describing polymers, which may be suitable as nitric oxide eluting polymers, are polymers comprising secondary amine groups (═N—H), such as L-PEI, or have a secondary amine (═N—H) as a pendant, such as aminocellulose.

The nitric oxide eluting polymer may comprise a secondary amine, either in the backbone or as a pendant, as described previously. This will make a good nitric oxide eluting polymer. The secondary amine should have a strong negative charge to be easy to load with nitric oxide. If there is a ligand close to the secondary amine, such as on a neighbor carbon atom to the nitrogen atom, with higher electronegativity than nitrogen (N), it is very difficult to load the polymer with nitric oxide. On the other hand, if there is a positive ligand close to the secondary amine, such as on a neighbor carbon atom to the nitrogen atom, the electronegativity of the amine will increase and thereby increase the possibility to load the nitric oxide elution polymer with nitric oxide.

In an embodiment the nitric oxide polymer may be stabilized with a salt. Since the nitric oxide eluting group, such as a diazeniumdiolate group, is negative, a positive counter ion, such as a cation, may be used to stabilize the nitric oxide eluting group. This cation may for example be selected from the group comprising any cation from group 1 or group 2 in the periodic table, such as Na⁺, K⁺, Li⁺, Be²⁺, Ca²⁺, Mg²⁺, Ba²⁺, and/or Sr²⁺. Different salts of the same nitric oxide eluting polymer have different properties. In this way a suitable salt (or cation) may be selected for different purposes. Examples of cationic stabilized polymers are L-PEI-NO—Na, i.e. L-PEI diazeniumdiolate stabilized with sodium, and L-PEI-NO—Ca, i.e. L-PEI diazeniumdiolate stabilized with calcium.

Another embodiment comprises mixing the nitric oxide eluting polymer, or a mixture of the nitric oxide eluting polymer and a carrier material, with an absorbent agent. This embodiment provides the advantage of an accelerated elution of nitric oxide since the polymer, or polymer mixture, via the absorbent agent, may take up the activating fluid, such as water or body fluid, much faster. In one example 80% (w/w) absorbent agent is mixed with the nitric oxide eluting polymer, or mixture of nitric oxide eluting polymer and carrier material, and in another embodiment 10 to 50% (w/w) absorbent agent is mixed with the nitric oxide eluting polymer, or mixture of nitric oxide eluting polymer and carrier material.

Since the elution of nitric oxide is activated by a proton donor, such as water, it may be an advantage to keep the nitric oxide eluting polymer, or mixture of nitric oxide eluting polymer and carrier material, in contact with said proton donor. If an indication requires an elution of nitric oxide during a prolonged period of time, a system is advantageous, which presents the possibility to keep the proton donor in contact with the nitric oxide eluting polymer, or mixture of nitric oxide eluting polymer and carrier material. Therefore, in still another embodiment, the elution of nitric oxide may be regulated by adding an absorbent agent. The absorbent agent absorbs the proton donor, such as water, and keeps the proton donor in close contact with the nitric oxide eluting polymer during prolonged periods of time. Said absorbent agent may be selected from the group comprising polyacrylates, polyethylene oxide, carboxymethylcellulose, and microcrystalline cellulose, cotton, and starch. This absorbent agent may also be used as a filling agent. In this case said filling agent may give the nitric oxide eluting polymer, or mixture of said nitric oxide eluting polymer and a carrier material, a desired texture.

This patch/pad may be in any suitable size, such as a suitable size for applying said patch/pad on a shoulder, or any other suitable area on a mammal. These sizes may for example vary from small, medium, and large sized patches/pads.

When the patch/pad is applied on an suitable area, the patch/pad starts to elute both NO and nicotine.

The elution of NO starts when the NO eluting polymer gets in contact with moisture, or water, in the form of, for example, sweat. Since NO has a vasodilating effect the transportation of nicotine is improved. The vasoconstrictive effect from nicotine is also counteracted by the vasodilating effect from NO. Therefore, this embodiment provides the advantages of promoting transportation of nicotine to the circulation of blood, and counteract the side effect of vasoconstrictive effect from nicotine.

Activation on NO release may be done by e.g. sweat, or a proton donor, such as water, sprayed onto the patch/pad immediately prior to use, or a proton donor bag configured for releasing the proton donor upon activation, e.g. by pushing onto the bag thus bursting (see below).

In another embodiment a nitric oxide eluting polymer is provided, and/or combined, with microencapsulated proton donor.

This may for example be done by first manufacture microcapsules, containing a proton donor, such as water or water containing liquid, in a state of the art manner. These microcapsules are then applied on the NO eluting polymer. The application of the microcapsules on the NO eluting polymer may for example be done by gluing, such as pattern gluing, or instead spinning the NO eluting polymer onto said microcapsules. In this way a device or a system, comprising NO eluting polymer and microencapsulated proton donor is manufactured. When the device or system is applied on the target area the device or system is compressed or squeezed. Said compression or squeezing results in breakage of the microcapsules. The NO eluting polymer is thus exposed to proton donor, and the elution of NO from the NO eluting polymer is initiated on the target area. In other embodiments the proton donor inside the microcapsules is released by heating or shearing the microcapsules until the microcapsules are ruptured.

In still another embodiment the microcapsules are formed into a film, tape, or sheath. Thereafter, a film, tape, or sheath of an NO eluting polymer is glued onto the film, tape, or sheath of microcapsules. Preferably the film, tape, or sheath of the NO eluting polymer is glued onto the film, tape, or sheath of the microcapsules in patterned way. The obtained pattern includes spaces where there is no glue, in which spaces the proton donor will be transported to the NO eluting polymer once the microcapsules are broken from compression or squeezing. When the proton donor gets in contact with the NO eluting polymer the elution of NO starts. Thus, the combination of film, tape, or sheath of microcapsules and NO eluting polymer may be applied on a target area. Thereafter the combination is compressed or squeezed, which results in that the target area is exposed to NO.

In yet another embodiment the NO eluting polymer is spun directly onto the film, tape, or sheath of microcapsules, containing proton donor. The combination of film, tape, or sheath of microcapsules and spun NO eluting polymer may be applied on a target area. Thereafter the combination is compressed or squeezed, which results in that the target area is exposed to NO.

In still another embodiment the device or system is provided with an activation indicator. This activation indicator indicates when the microcapsules are satisfyingly broken, hence when the NO eluting polymer is subjected to enough proton donor to elute an efficient amount of NO. This activation indicator may for example be obtained by coloring the proton donor that is trapped inside the microcapsules. When the microcapsules are broken the colored proton donor escapes the microcapsules and the color gets visualized while efficiently wetting the NO eluting polymer. Another way of obtaining an activation indicator is to choose to manufacture the microcapsules in a material, or choose a wall thickness of said microparticles, that creates a sound when the microcapsules break. It is also possible to admix a scent in the proton donor, contained in the microcapsules. This results in that the user of the device or system may smell the scent when the proton donor escapes from the microcapsules after breakage thereof.

In another embodiment a substance that changes color when it comes in contact with water can be incorporated in the device. Thus when the water capsules or water bag breaks the material changes color, thereby indicating that the material is activated.

In another embodiment the device or system only allows NO-elution in one direction. In this kind of embodiment one side of the device has low permeability, or substantially no permeability, to nitric oxide. This may also be accomplished by applying a material on one side of the device according to the invention that is not permeable to NO. Such materials may be chosen from the group comprising common plastics, such as fluoropolymers, polyethylene, polypropylene, polyacrylonitrile, polyurethane, polyvinylacetates, polylacticacids, starch, cellulose, polyhydroxyalkanoates, polyesters, polycaprolactone, polyvinylalcohol, polystyrene, polyethers, polycarbonates, polyamides, poly(acrylic acid), Carboxy Methyl Cellulose (CMC), protein based polymers, gelatine, biodegradable polymers, cotton, and latex, or any combinations of these. This embodiment is also easy to manufacture as the NO eluting polymer, e.g. L-PEI (or nitric oxide eluting polymer and carrier material, which will be explained in more detail below) may be electro or gas-jet spun onto the surface of the device according to the invention of e.g. the mentioned plastics, latex, or cotton.

In still another embodiment the device is provided with one membrane, which is permeable to nitric oxide, on a first side of the device, and another membrane, which has low permeability or substantially no permeability to nitric oxide, on a second side of said device. This embodiment provides the possibility to direct the elution to said first side of the device, while the elution of nitric oxide is substantially prevented from said second side. Thereby, a greater amount of nitric oxide will reach the intended area to be treated.

The activation of the nitric oxide eluting polymer may be accomplished by contacting said polymer with a suitable proton donor. In one embodiment the proton donor may be selected from the group comprising water, body fluids (blood, lymph, bile, etc.), alcohols (methanol, ethanol, propanols, buthanols, pentanols, hexanols, phenols, naphtols, polyols, etc.), aqueous acidic buffers (phosphates, succinates, carbonates, acetates, formats, propionates, butyrates, fatty acids, amino acids, etc.), or any combinations of these.

By adding a surfactant in the proton donor one can facilitate the wetting of the device. The surfactant lowers the surface tension and the activating fluid is easily transported throughout the device.

According to another embodiment the patch/pad is made of, or comprise, nanofilaments, e.g. made by electro or gas jet spinning, comprising nicotine. According to a further embodiment the patch/pad comprises microspheres eluting NO in use. Preferably the three aforementioned embodiments employ L-PEI material loaded with NO.

When the NO-eluting patch/pad, comprising nicotine, is treated with or gets in contact with the moisture, in form of secreted sweat, the NO-eluting patch/pad, comprising nicotine, starts to release NO and nicotine to the area to be treated. Alternatively the device is moisturized or wetted immediately prior to application or use for controlling or activating the NO release.

In another embodiment a patch/pad is covered on the inside with NO-eluting nanoparticles, or microspheres. These nanoparticles, or microspheres, may be formed from the NO-eluting polymers. They may also be encapsulated in any suitable material, such as polyethylene, polypropylene, polyacrylonitrile, polyurethane, polyvinylacetates, polylacticacids, starch, cellulose, polyhydroxyalkanoates, polyesters, polycaprolactone, polyvinylalcohol, polystyrene, polyethers, polycarbonates, polyamides, polyolefins, poly(acrylic acid), Carboxy Methyl Cellulose (CMC), protein based polymers, gelatine, biodegradable polymers, cotton, and latex, or any combinations of these. When the nanoparticles, or microspheres, according to this embodiment, gets in contact with the secreted moisture, in form of sweat, on the inside of the patch/pad, they start to elute NO on the area to be treated.

In the context of the present invention the term “encapsulating” is intended to be interpreted as fixating the nitric oxide eluting polymer in a three dimensional matrix such as a foam, a film, a nonwoven mat of nanofibers, fibers, or other materials with the capability to fixate the NO eluting polymer, or enclosing the nitric oxide eluting polymer in any suitable material.

In yet another embodiment the patch/pad contains a small water bag or sealed water sponge. This water bag or sealed water sponge is used to activate the elution of NO from the NO-eluting nanoparticles, or microspheres. Persons that do not easily sweat may be helped by the use of this embodiment.

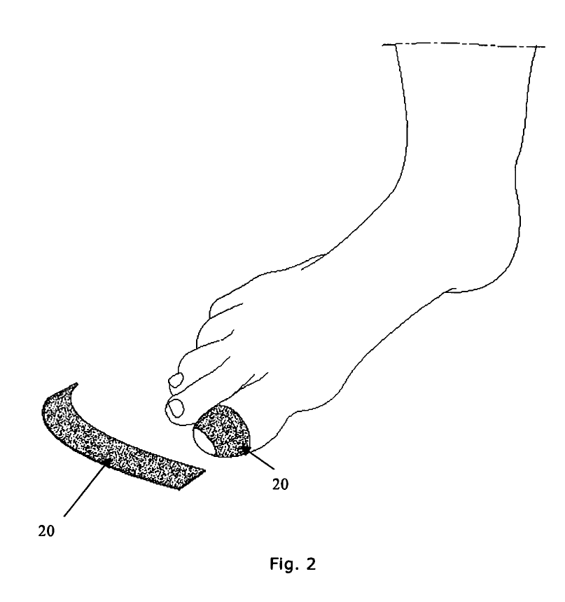

In still another embodiment of the device, it may be manufactured in the form of a polyurethane, or polyethylene, tape or coating, according to FIG. 2. This polyurethane tape or coating may easily be wrapped around a suitable body part. At least the side facing the body part may be covered with NO-eluting nanoparticles, or microspheres, or nanofilament of NO-eluting L-PEI. When these particles or filaments get in contact with the moisture, in form of sweat, on the inside of the tape or coating, the elution of NO starts simultaneously as elution of nicotine.

Of course, in other embodiments of the invention, the patch/pad or tape/coating may be manufactured by any other suitable material, such as polyethylene, polypropylene, polyacrylonitrile, polyurethane, polyvinylacetates, polylacticacids, starch, cellulose, polyhydroxyalkanoates, polyesters, polycaprolactone, polyvinylalcohol, polystyrene, polyethers, polycarbonates, polyamides, polyolefins, poly(acrylic acid), Carboxy Methyl Cellulose (CMC), protein based polymers, gelatine, biodegradable polymers, cotton, and latex, or any combinations of these. The NO-eluting polymer may be integrated in, spun together with, or spun on top of, any of these materials in all of the embodiments, while still containing nicotine in an eluting form.

In another embodiment these nanoparticles, or microspheres, may be integrated in a soluble film that disintegrates on the inside of the patch/pad or tape/coating, in order to elute NO at the area of interest when the soluble film gets in contact with the moisture, in the form of sweat or from the proton donor bag or sealed proton donor sponge, on the area to be treated.

When placed on an area to be treated the device provides vasodilatation, which vasodilatation counteract the vasoconstriction of the other active component, such as nicotine, comprised in the patch/pad and/or tape/coating.

In another embodiment the device only allows NO- and elution of a drug, such as nicotine, in one direction. In this kind of embodiment one side of the device has low permeability, or substantially no permeability, to nitric oxide and said drug. This may also be accomplished by applying a material on one side of the device according to the invention that is not permeable to NO and said drug. Such materials may be chosen from the group comprising common plastics, such as fluoropolymers, polyethylene, polypropylene, polyacrylonitrile, polyurethane, polyvinylacetates, polylacticacids, starch, cellulose, polyhydroxyalkanoates, polyesters, polycaprolactone, polyvinylalcohol, polystyrene, polyethers, polycarbonates, polyamides, poly(acrylic acid), Carboxy Methyl Cellulose (CMC), protein based polymers, gelatine, biodegradable polymers, cotton, and latex, or any combinations of these. This embodiment is also easy to manufacture as the NO eluting polymer, e.g. L-PEI (or nitric oxide eluting polymer and carrier material, which will be explained in more detail below) may be electro or gas-jet spun onto the surface of the device according to the invention of e.g. the mentioned plastics, latex, or cotton.

The patch/pad or tape/coating may be turned outside in, or in any other way arranged to protect the NO and drug eluting side, after manufacturing to protect the NO and drug eluting polymer during packaging, transport and prior to use from external influences, being e.g. mechanical (abrasion of the polymer), chemical (moisture deactivating the device prior to use) etc.

In still another embodiment the device is provided with one membrane, which is permeable to nitric oxide and drug, on a first side of the device, and another membrane, which has low permeability or substantially no permeability to nitric oxide and drug, on a second side of said device. This embodiment provides the possibility to direct the elution to said first side of the device, while the elution of nitric oxide and drug is substantially prevented from said second side. Thereby, a greater amount of nitric oxide and drug will reach the intended area to be treated.

In another embodiment the NO-eluting device is acting as a booster for drug eluting patches, e.g. pharmaceuticals, vitamins, nitroglycerin, diclofenac etc. This embodiment presents a device with the advantage of combining two therapeutic treatments, of significant value, in one treatment. Hence, a synergetic effect may be achieved by such devices, when NO is eluted from the device. NO has a vasodilatory effect on the region where the device having the combination compound actuates. Vasodilated tissue is more susceptible to certain medications and thus more easily treated by the medical preparations and still NO has in addition to that the anti-inflammatory, anti-bacterial etc. effect. Hence, an unexpected surprisingly effective treatment is provided.

Not only does the nitric oxide vasodilate, or counteract vasoconstriction, at the application site, to thereby make it easier for a drug to enter the circulation system, the nitric oxide also boosts the effect of a drug at the target area of said drug, i.e. a synergic effect is obtained by the use of nitric oxide in combination with drugs. In another embodiment of the device the fibers, nanoparticles, or microspheres may be integrated, and combined with other drugs, such as nicotine, in a gel, cream, or foam, that may either be in a smearing or compressed structure. The elution of NO and nicotine may then be initiated by applying a water soaked patch on said gel. The fibers, nanoparticles, or microspheres may also be integrated in a hydrogel, which is mixed directly before use. This embodiment has the advantage of being able to penetrate pockets and corners in the skin for closer elution of NO on the area to be treated.

In still another embodiment the nitric oxide eluting polymer, such as powder, nanoparticles or microspheres, can be incorporated in said foam. The foam may have an open cell structure, which facilitates the transport of the proton donor to the nitric oxide eluting polymer. The foam can be of any suitable polymer such as polyethylene, polypropylene, polyacrylonitrile, polyurethane, polyvinylacetates, polylacticacids, starch, cellulose, polyhydroxyalkanoates, polyesters, polycaprolactone, polyvinylalcohol, polystyrene, polyethers, polycarbonates, polyamides, poly(acrylic acid), Carboxy Methyl Cellulose (CMC), protein based polymers, gelatine, biodegradable polymers, cotton, polyolefins, and latex, or any combinations of these, or latex.

In another embodiment, according to above, the device is in the form of a cream, a gel or a combination of the two. Since the nitric oxide eluting polymer is activated by proton donors the nitric oxide eluting polymer has to be separate from the proton donor until one wants to initiate the elution of nitric oxide, i.e. use the device. One way to accomplish this is to have a syringe with two separate containers. In one container you have a proton donor-based gel and in the other a non proton donor-based gel, comprising the nitric oxide eluting polymer. Upon using the device the two gels are squeezed from the syringe and mixed together, the proton donor in the first gel comes in contact with the nitric oxide eluting polymer in the second gel and the elution of nitric oxide starts.

In other embodiments the drug is nicotine, diclofenac or cortisone. Other active substances may of course also be possible to include in the devices, and these examples are solely intended to be exemplifying the present invention, and not limiting the scope of the present invention in any way.

In another embodiment the device may act to improve absorption of active dermatological drugs, for example Non-Steroidal Anti-Inflammatory Drugs (NSAID), such as diclofenac, ibuprofen, aspirin, naproxen, COX-2 inhibitors, choline magnesium trisalicylate, diflunisal, salsalate, fenoprofen, flurbiprofen, ketoprofen, oxaprozin, indomethacin, sulindac, tolmetin, meloxicam, piroxicam, meclofenamate, mefenamic acid, nabumetone, etodalac, ketorolac, celecoxib, valdecoxib, and rofecoxib; steroids, such as cortisone, prednisone, methylprednisolone, prednisolone, vitamin D, estrogen, cholestrol, beclomethasone, flunisolide, fluticasone, triamcinolone, desonide, clobetasol, alclometasole, desoximetasone, betamethasone, halcinonide and dexamethasone; pain reliefs, such as motrin, feldene, naprosyn, lidocaine, and prilocaine; and other substances, such as indinavirsulfate, finasteride, aprepitant, montelukast sodium, alendronate sodium, rofecoxib, rizatriptan benzoate, simvastatin, finasteride, ezetimibe, caspofungin acetate, ertapenem sodium, dorzolamide hydrochloride, timolol maleate, losartan potassium, and hydrochlorotiazide; etc. In this embodiment the elution of NO does not counteract a side effect in form of vasoconstriction, but improves absorption of active dermatological substances through the vasodilating effect of NO. This is also true in respect of insulin as a drug. When the device gets in contact with water or moisture, according to above, the devices starts to elute NO. Thus, according to above, a vasodilating effect is obtained. This vasodilating effect improves the absorption of said active dermatological substances. This embodiment has the advantage of presenting an improvement in respect of the effect of said dermatological substances, as said dermatological substances obtain a faster and more effective access to the target tissue.

The device elutes nitric oxide (NO) from said eluting polymer in a therapeutic dose, such as between 0.001 to 5000 ppm, such as 0.01 to 3000 ppm, such as 0.1 to 1000 ppm, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90 91, 92, 93, 94, 95, 96, 97, 98, 99, or 100 ppm. The concentration may vary widely depending on where the concentration is measured. If the concentration is measured close to the actual NO eluting polymer the concentration may be as high as thousands of ppm, while the concentration inside the tissue in this case often is considerably lower, such as between 1 to 1000 ppm.

Three important factors in controlling and regulating the elution of nitric oxide from a nitric oxide eluting polymer are how quickly a proton donor comes in contact with the nitric oxide releasing polymer, such as a diazoliumdiolate group, the acidity of the environment surrounding the nitric oxide eluting polymer, and the temperature of the environment surrounding the nitric oxide releasing polymer (higher temperature promotes elution of nitric oxide).

In the embodiments it may be suitable to control or regulate the time span of NO release from the device according to the invention. This may be accomplished by integrating other polymers or materials in said device. These polymers or materials may be chosen from any suitable material or polymer, such as polyethylene, polypropylene, polyacrylonitrile, polyurethane, polyvinylacetates, polylacticacids, starch, cellulose, polyhydroxyalkanoates, polyesters, polycaprolactone, polyvinylalcohol, polystyrene, polyethers, polycarbonates, polyamides, polyolefins, poly(acrylic acid), Carboxy Methyl Cellulose (CMC), protein based polymers, gelatine, biodegradable polymers, cotton, and latex, or any combinations of these.

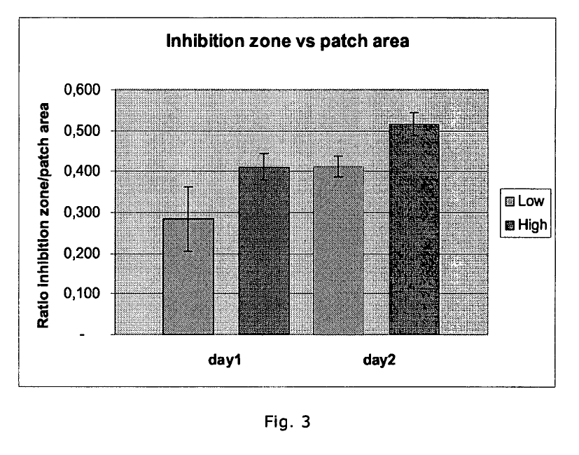

In one embodiment a nitric oxide eluting polymer, such as L-PEI-NO, is mixed with a carrier polymer to slow down or prolong the elution of nitric oxide. Also, in another embodiment, the nitric oxide eluting polymer may be mixed with more than one carrier polymer, whereby be elution or release may be tailor made to fit specific needs. Such a need may for example be a low elution during a first period of time, when the environment of the nitric oxide eluting polymer is hydrophobic, and a faster elution during a second period of time, when the environment of the nitric oxide eluting polymer has been altered to be more hydrophilic. This may for example be accomplished by using biodegradable polymers, whereby a low elution during a first period of time is obtained, after which, when the hydrophobic polymer has been dissolved, the hydrophilic polymer provides a higher elution of nitric oxide. Thus, a more hydrophobic carrier polymer will give a slower elution of nitric oxide, since the activating proton donor, such as water or body fluid, will penetrate the carrier polymer slower. On the other hand, a hydrophilic polymer acts the opposite way. One example of a hydrophilic polymer is polyethylene oxide, and one example of an hydrophobic polymer is polystyrene. These carrier polymers may be mixed with the nitric oxide eluting polymer and then electrospun to suitable fibers. The skilled person in the art knows which other polymers may be used for similar purposes. FIG. 3 illustrates two elution profiles (NO concentration vs. time) for two different polymer mixtures; a nitric oxide eluting polymer mixed with a hydrophilic carrier polymer in an acidic environment (A), and a nitric oxide eluting polymer mixed with a hydrophobic carrier polymer in a neutral environment (B).

In one embodiment this carrier polymer is substituted by another material with hydrophobic or hydrophilic properties. Therefore, the term “carrier material” in the present context should be interpreted to include carrier polymers and other materials with hydrophilic or hydrophobic properties.

In another embodiment the elution of nitric oxide from a nitric oxide eluting polymer, such as L-PEI-NO, is influenced by the presence of protons. This means that a more acidic environment provides a quicker elution of nitric oxide. By activating the nitric oxide eluting polymer, or mixture of nitric oxide eluting polymer and carrier material, with an acidic fluid, such as an ascorbic acid solution, the elution of nitric oxide may be accelerated.

The carrier polymers and carrier materials mentioned above may affect other characteristics than the regulation of nitric oxide elution. An example of such characteristic is mechanical strength.

In respect of the carrier polymers or carrier materials, the NO-eluting polymer may be integrated in, spun together with, or spun on top of, any of these materials in all of the embodiments. This spinning includes electro spinning, air spinning, dry spinning, wet spinning, melt spinning, and gel spinning. In this way, one may manufacture fibers of a polymer mixture, comprising a nitric oxide eluting polymer and a carrier polymer, or a carrier material, with predefined nitric oxide eluting characteristics. These characteristics may be tailor made for different elution profiles in different applications.

The NO-eluting polymers in the devices may be combined with silver, such as hydroactivated silver, for instance eluting effective silver ions. The integration of silver in the devices gives the healing process an extra boost. Preferably the silver is releasable from the devices in the form of silver ions. The integration of silver in the device may present several advantages. One example of such an advantage is that the silver may keep the device in itself free from bacteria or viruses, while the nitric oxide eluting polymer elutes the therapeutic dosage of nitric oxide to the target site.

The device may be manufactured by, for example electro spinning of L-PEI or other polymers comprising L-PEI or being arranged in combination with L-PEI. L-PEI is the charged at a characteristic voltage, and a fine jet of L-PEI releases as a bundle of L-PEI polymer fibers. This jet of polymer fibers may be directed to a surface to be treated. The surface to be treated may for example be any suitable material in respect of a device, comprising any other active component, such as nicotine, cortisone, diclofenac, etc. The electro spun fibers of L-PEI then attach on said material and form a coating/layer of L-PEI on the device according to the invention.

It is of course possible to electro spin the other NO-eluting polymers, according to above, on the device according to the invention while still being inside the scope of the present invention.

In one embodiment the NO-eluting polymers are electro spun in such way that pure NO-eluting polymer fibers may be obtained.

It is also within the scope of the present invention to electro spin a NO-eluting polymer together with other suitable polymer/polymers.

Gas spinning, air spinning, wet spinning, dry spinning, melt spinning, or gel spinning, of said NO-eluting polymers onto the device is also within the scope of the present invention, offering certain advantages in comparison to other manufacturing methods.

The manufacturing process presents the advantages of large contact surface of the NO-eluting polymer fibers with the area to be treated, effective use of NO-eluting polymer, and a cost effective way of producing the device.

Hereinafter, some potential uses of the present invention are described:

A method of therapeutically treating a mammal, such as a human, by means of a device that comprises a drug and a nitric oxide (NO) eluting polymer configured for eluting a therapeutic dosage of nitric oxide (NO) when used for therapeutic treatment, comprising

exposing a treatment site in or on a body to said drug and to said nitric oxide when said polymer in use elutes nitric oxide (NO), such that that treatment site is more susceptible to said drug than without said eluted nitric oxide (NO), whereby the therapeutic treatment is rendered more effective.

A method according to the aforementioned method, wherein said site is an extremity of a body, and wherein said method comprises applying a condom/sheath, a sock, a patch/pad, a tape/coating gel, cream, foam, hydrogel or combinations thereof, comprising said nitric oxide (NO) eluting polymer and said drug, to said extremity for said exposure.

Use of nitric oxide (NO) to boost the effect of a drug, wherein said nitric oxide (NO) and said drug are, preferably as a compound, comprised in a medical device, wherein said nitric oxide (NO) in said use is eluted, preferably in pure form, from a nitric oxide (NO) eluting polymer, wherein said nitric oxide (NO) eluting polymer is comprised in said device in a suitable form and configured for eluting a therapeutic dosage of nitric oxide (NO), comprising exposing a treatment site in or on a mammal body, such as a human body, to said drug and to said nitric oxide when said polymer in use elutes nitrogen oxide (NO), such that that treatment site is more susceptible to said drug for boosting the effect of said drug.

The invention may be implemented in any suitable form. The elements and components of the embodiments according to the invention may be physically, functionally, and logically implemented in any suitable way. Indeed, the functionality may be implemented in a single unit, in a plurality of units, or as part of other functional units.

Although the present invention has been described above with reference to specific embodiments, it is not intended to be limited to the specific form set forth herein. Rather, the invention is limited only by the accompanying claims and, other embodiments than the specific above are equally possible within the scope of these appended claims.

In the claims, the term “comprises/comprising” does not exclude the presence of other elements or steps. Furthermore, although individually listed, a plurality of means, elements or method steps may be implemented. Additionally, although individual features may be included in different claims, these may possibly advantageously be combined, and the inclusion in different claims does not imply that a combination of features is not feasible and/or advantageous. In addition, singular references do not exclude a plurality. The terms “a”, “an”, “first”, “second” etc do not preclude a plurality. Reference signs in the claims are provided merely as a clarifying example and shall not be construed as limiting the scope of the claims in any way.

What is claimed is:

1. A device configured to therapeutically treat a target site in or on an animal body,
wherein said device comprises a drug and a nitric oxide (NO) eluting polymer, wherein said nitric oxide (NO) eluting polymer is configured to elute a therapeutic dosage of nitric oxide (NO), wherein said device is configured to expose the target site to said drug and to said nitric oxide when said polymer elutes nitric oxide (NO), wherein said treatment site is more susceptible to said drug and wherein said NO, when eluted, boosts the effect of said drug at said target site.

2. The device according to claim 1, wherein said nitric oxide (NO) eluting polymer comprises diazeniumdiolate groups, S-nitrosylated groups, O-nitrosylated groups, or any combination thereof.

3. The device according to claim 1, wherein said nitric oxide (NO) eluting polymer is L-PEI (linear polyethyleneimine).

4. The device according to claim 1, wherein said nitric oxide eluting polymer is selected from the group consisting of amino cellulose, amino dextrans, chitosan, aminated chitosan, polyethyleneimine, PEI-cellulose, polypropyleneimine, polybutyleneimine, polyurethane, poly(buthanediol spermate), poly(iminocarbonate), polypeptide, Carboxy Methyl Cellulose (CMC), polystyrene, poly(vinyl chloride), polydimethylsiloxane, and any combination thereof, and wherein said polymer is grafted to an inert backbone selected from the group consisting of a polysaccharide backbone and a cellulosic backbone.

5. The device according to claim 1, wherein said device is a condom/sheath, a sock, a patch/pad, or a tape/coating, and is adapted to be applied on or at said target site.

6. The device according to claim 1, wherein said polymer comprises silver.

7. The device according to claim 1, wherein said polymer is in the form of nanoparticles or microspheres.

8. The device according to claim 7, wherein said nanoparticles or microspheres, are integrated in a gel, hydrogel, cream, foam, or any combination thereof.

9. The device according to claim 7, wherein said nanoparticles or microspheres, are encapsulated in a carrier material, selected from the group consisting of polyethylene, polypropylene, polyacrylonitrile, polyurethane, polyvinylacetates, polylacticacids, starch, cellulose, polyhydroxyalkanoates, polyesters, polycaprolactone, polyvinylalcohol, polystyrene, polyethers, polycarbonates, polyamides, polyolefins, poly(acrylic acid), Carboxy Methyl Cellulose (CMC), protein based polymers, gelatine, biodegradable polymers, cotton, latex, and any combination thereof.

10. The device according to claim 1, further comprising a proton donor bag, sealed proton donor sponge, or microencapsulated proton donor.

11. The device according to claim 1, wherein said device is partly disintegrable when subjected to a proton donor.

12. The device according to claim 10, wherein said proton donor is selected from the group consisting of water, blood, lymph, bile, methanol, ethanol, propanols, butanols, pentanols, hexanols, phenols, naphtols, polyols, phosphates, succinates, carbonates, acetates, formats, propionates, butyrates, fatty acids, amino acids, and any combination thereof.

13. The device according to claim 1, wherein said nitric oxide eluting polymer comprises a secondary amine in the backbone or a secondary pendant amine.

14. The device according to claim 13, wherein a positive ligand is located on a neighbor carbon atom to the secondary amine.

15. The device according to claim 1, further comprising an absorbent agent.

16. The device according to claim 15, wherein said absorbent agent is selected from the group consisting of polyacrylate, polyethylene oxide, Carboxy Methyl Cellulose (CMC), microcrystalline cellulose, cotton, starch, and any combination thereof.

17. The device according to claim 1, further comprising a cation for stabilizing the nitric oxide eluting polymer.

18. The device according to claim 17, wherein said cation is selected from the group consisting of Na⁺, K⁺, Li⁺, Be²⁺, Ca²⁺, Mg²⁺, Ba²⁺, Sr²⁺, and any combination thereof.

19. The device according to claim 1, wherein said drug is selected from the group consisting of a pharmaceutical agent, a vitamin, nicotine, nitroglycerin, a Non-Steroidal Anti-Inflammatory Drugs (NSAID), a steroid, an analgesic, indinavirsulfate, finasteride, aprepitant, montelukast sodium, alendronate sodium, rofecoxib, rizatriptan benzoate, simvastatin, finasteride, ezetimibe, caspofungin acetate, ertapenem sodium, dorzolamide hydrochloride, timolol maleate, losartan potassium, and hydrochlorotiazide.

20. The method of claim 19, wherein said NSAID is selected from the group consisting of diclofenac, ibuprofen, aspirin, naproxen, a COX-2 inhibitor, choline magnesium trisalicylate, diflunisal, salicylate, fenoprofen, flurbiprofen, ketoprofen, oxaprozin, indomethacin, sulindac, tolmetin, meloxicam, piroxicam, meclofenamate, mefenamic acid, nabumetone, etodalac, ketorolac, celecoxib, valdecoxib and rofecoxib.

21. The method of claim 19, wherein said steroid is selected from the group consisting of cortisone, prednisone, methylprednisolone, prednisolone, vitamin D, estrogen, cholesterol, beclomethasone, flunisolide, fluticasone, triamcinolone, desonide, clobetasol, alclometasole, desoximetasone, betamethasone, halcinolide and dexamethasone.

22. The method of claim 19, wherein said analgesic is selected from the group consisting of motrin, feldene, naprosyn, lidocaine and prilocaine.

23. A manufacturing process for a device configured for therapeutic treatment according to claim 1, comprising:
selecting a plurality of nitric oxide eluting polymeric particles selected from the group consisting of nanofibers, nanoparticles or microspheres;
selecting a drug; and
deploying said nitric oxide eluting particles and said drug into a suitable form, or as a coating onto a carrier, to form said device,
wherein said deploying comprises electro spinning, gas spinning, air spinning, wet spinning, melt spinning, or gel spinning of said particles.

24. A manufacturing process according to claim 23, further comprising:
selecting a nitric oxide (NO) eluting polymer configured to elute a therapeutic dosage of nitric oxide (NO) from said nitric oxide eluting polymeric particles;
selecting a carrier material configured to regulate the elution of said therapeutic dosage of nitric oxide (NO);
incorporating the NO-eluting polymer with said carrier material into a nitric oxide (NO) eluting material, wherein said carrier material regulates the elution of said therapeutic dosage of nitric oxide (NO), and
deploying said nitric oxide eluting material into a suitable form, or as a coating onto a carrier, to form at least a part of said device, wherein said device is configured to expose a target site to said nitric oxide, when said NO-eluting polymer elutes nitric oxide (NO), and to said drug.

25. The manufacturing process according to claim 24, wherein said selecting said nitric oxide (NO) eluting polymer comprises selecting a plurality of nitric oxide (NO) eluting polymeric particles selected from the group consisting of nanofibers, nanoparticles and microspheres.

26. The manufacturing process according to claim 24, wherein said incorporating said NO-eluting polymer with said carrier material comprises integrating said NO-eluting polymer in said carrier material, spinning said NO-eluting polymer together with said carrier material, or spinning said NO-eluting polymer on top of said carrier material.

27. The manufacturing process according to claim 23, further comprising
microencapsulating a proton donor in microcapsules, and
applying the microcapsules to said nitric oxide (NO) eluting material.

28. The manufacturing process according to claim 27, wherein said applying comprises pattern gluing or spinning the NO eluting material onto said microcapsules.

29. The manufacturing process according to claim 28, further comprising:
forming the microcapsules into a first film, tape, or sheath;
forming a second film, tape, or sheath of said NO eluting material; and
gluing the first film, tape, or sheath of microcapsules to said second film, tape, or sheath of said NO eluting material.

30. The manufacturing process according to claim 29, wherein said gluing comprises patterned gluing, wherein a pattern is obtained that includes glue-free spaces.

31. The manufacturing process according to claim 27, further comprising forming the microcapsules into a first film, tape, or sheath, and directly spinning the NO eluting material onto the film, tape, or sheath of microcapsules, wherein said film, tape or sheath includes the proton donor.

32. The manufacturing process according to claim 27, further comprising providing an activation indicator configured to indicate when the microcapsules are broken wherein the NO eluting material is subjected to said proton donor to elute NO.

33. The manufacturing process according to claim 32, wherein said providing an activation indicator comprises providing a coloring agent inside the microcapsules.

34. The manufacturing process according to claim 32, wherein said providing an activation indicator comprises selecting a material for the microcapsules, or choosing a wall thickness of said microcapsules, that creates a sound when the microcapsules break.

35. The manufacturing process according to claim 32, wherein said providing an activation indicator comprises admixing a scent material into the microcapsules.

36. The manufacturing process according to claim 32, wherein said providing an activation indicator comprises providing a substance that changes color when it comes in contact with the proton donor.

37. A method of enhancing the effect of a drug at a treatment site in a mammal, comprising contacting said site with said drug and a nitric oxide (NO) eluting polymer configured for eluting a therapeutic dosage of nitric oxide (NO), wherein said polymer elutes said nitric oxide (NO), wherein the treatment site is more susceptible to said drug than in the absence of said eluted nitric oxide (NO), thereby rendering the treatment more effective.

38. The method of claim 37, wherein said drug and said nitric oxide are contained within a device or composition.

39. The method according to claim 38, wherein said site is an extremity, and wherein said device or composition is a condom/sheath, a sock, a patch/pad, a tape/coating gel, cream, foam, hydrogel or any combination thereof.
